(19) United States
(12) Patent Application Publication
(10) Pub. No.: US 2012/0142951 A1
(43) Pub. Date: Jun. 7, 2012

(54) METHOD OF PREPARING alpha-SULFO FATTY ACID ALKYL ESTER ALKANOLAMINE SALT

(76) Inventors: Hideo Andoh, Tokyo (JP); Mikako Sakurai, Tokyo (JP)

(21) Appl. No.: 13/263,221

(22) PCT Filed: Apr. 7, 2010
(86) PCT No.: PCT/JP2010/002555

§ 371 Date: Nov. 9, 2011

(30) Foreign Application Priority Data
Apr. 10, 2009 (JP) 2009-095937

(51) Int. Cl. C07C 309/62

(52) U.S. Cl. 554/94

(57) ABSTRACT

The invention provides a method for the preparation of α-sulfo fatty acid alkylester alkanolamine salt comprising: sulfonating a fatty acid alkyl ester to obtain a sulfonated product, reacting an alcohol and a hydrogen peroxide with the sulfonated product to obtain a reactive product, adding an aqueous alkanolamine solution to the resultant reactive product to obtain a neutralized product having a pH of 2 to 6.5; condensing the neutralized product to obtain an aqueous paste containing α-sulfo fatty acid alkylester alkanolamine salt.

According to the present invention, color deterioration can be inhibited at the time of condensation during preparation of alkanolamine salt of α-sulfo fatty acid alkylester.

### TECHNICAL FIELD

The present invention relates to a method of preparing α-sulfo fatty acid alkyl ester alkanolamine salt.

Priority is claimed on Japanese Patent Application No. 2009-095937, filed Apr. 10, 2009, the content of which is incorporated herein by reference.

### BACKGROUND ART

A preparation of α-sulfo fatty acid alkyl ester salt, which is one kind of anionic surfactant, is highly regarded as a cleaning material in terms of having a superior cleaning ability and good biodegradability.

The α-sulfo fatty acid alkyl ester salt is prepared as an aqueous paste by contacting a fatty acid alkyl ester with a sulfonating gas, followed by performing an aging process, and performing an esterification process by adding a lower alcohol to obtain a sulfonated product, which is then neutralized in an aqueous alkali solution. The aqueous paste is normally subjected to heating process so as to condense the α-sulfo fatty acid alkylester salt. These aqueous pastes of the α-sulfo fatty acid alkyl ester salt are also normally subjected to processes such as cooling, solidification, grinding and assembly, and then blended into a granulated detergent composition as a granulate. The α-sulfo fatty acid alkyl ester salt is mainly used as a sodium salt in these uses.

There is a problem in that coloration easily occurs at the time of sulfonation or condensation in α-sulfo fatty acid alkyl ester salt. Since the coloration when α-sulfo fatty acid alkyl ester salt is used as a detergent for clothing materials is undesirable, bleaching is normally performed during the preparation of the α-sulfo fatty acid alkyl ester salt.

Patent Literature 1 describes a method where the pH of an aqueous paste is regulated to 4.5 to 6.5 during the preparation of the α-sulfo fatty acid alkyl ester salt powder, a condensed product is heated to a temperature of 90 to 140° C. at the exit of a condensing dryer, to thereby inhibit deterioration of color at the time of condensation.

### Patent Literature

[PTL 1] Pamphlet of International Publication No. 2008/078609

### SUMMARY OF INVENTION

### Technical Problem

Recently, demand for a liquid detergent composition has increased. It is thought that when α-sulfo fatty acid alkyl ester salt is mixed with a liquid detergent, alkanolamine salt is more suitable than sodium salt. However, according to the research of the present inventors, the alkanolamine salt, in particular triethanolamine salt easily suffers from color deterioration in comparison to sodium salt at the time of condensation.

The method of PTL 1 is very efficient in the case where α-sulfo fatty acid alkyl ester salt forms a sodium salt, but has very low effectiveness in the case of alkanolamine salt.

Further, an aqueous paste of α-sulfo fatty acid alkyl ester salt contains an alcohol at the time of preparation thereof. However, when the aqueous paste is used as a liquid detergent, from in terms of handling, the alcohol in the aqueous paste should be reduced to a concentration of about 2% or less, so that a higher concentration is needed.

Therefore, there is a demand for a technique in which when an alkanolamine salt, in particular triethanolamine salt, is prepared, color deterioration at the time of condensation can be effectively inhibited.

The invention has been made from the viewpoint of these problems, and provides a preparation method capable of inhibiting color deterioration at the time of condensation when the α-sulfo fatty acid alkyl ester alkanolamine salt is prepared.

### Solution to Problem

As a result of carrying out extensive research, the inventors found the following. After sulfonating fatty acid ester, alcohol and hydrogen peroxide were added thereto and subjected to a bleaching process. To the obtained bleaching product, an aqueous alkanolamine solution was added so as to obtain a specific pH value and the bleaching product was partially neutralized, whereby color deterioration at the time of condensation could be prevented, thus completing the invention.

The invention to solve the problems relates to a method of preparing α-sulfo fatty acid alkyl ester alkanolamine salt. The invention relates to a preparation method having processes of (1) sulfonating a fatty acid alkyl ester to obtain a sulfonated product, (2) reacting alcohol and hydrogen peroxide with the sulfonated product to obtain a reactive product, (3) adding the aqueous alkanolamine solution to the reactive product to obtain a neutralization product having a pH of 2 to 6.5, and (4) condensing the neutralization product to obtain an aqueous paste containing α-sulfo fatty acid alkyl ester alkanolamine salt.

### Advantageous Effects of Invention

According to the invention, when α-sulfo fatty acid alkyl ester alkanol amine salt is prepared, color deterioration at the time of condensation can be inhibited.

### BRIEF DESCRIPTION OF DRAWINGS

FIG. 1 is a graph showing a result of Test Example 1.

FIG. 2 is a graph showing a result of Test Example 2.

### DESCRIPTION OF EMBODIMENTS

Hereinafter, the invention will be described in more detail.

Process (1)

In process (1), a fatty acid alkyl ester is sulfonated to obtain a sulfonated product.

Examples of the fatty acid alkyl ester include one represented by the following formula (I).

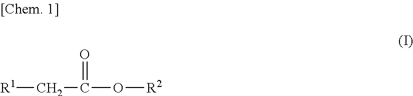

Wherein, R¹ represents a straight or branched alkyl group or alkenyl group having 8 to 20 carbon atoms, R² represents a straight or branched alkyl group having 1 to 4 carbon atoms.

Wherein, R¹ may be a straight or branched chain, of which the number of carbon atoms is preferably 10 to 18, more preferably 10 to 16.

R² may be a straight or branched chain, of which the number of carbon atoms is preferably 1 to 3, more preferably 1.

The “aliphatic” of the specification has a relative meaning to aromatic, and means a group and a compound having no aromatic properties.

An “alkyl group” includes a straight and branched monovalent saturated hydrocarbon group, unless otherwise specified.

Examples of the fatty acid alkyl ester include a fatty acid alkyl ester derived from animal-based fat such as beef fat, fish oil, lanolin; a fatty acid alkyl ester derived from plant-based fat such as coconut oil, palm oil, soy bean oil; synthetic fatty acid alkyl ester derived from oxo process of α-olefin, and is not particularly limited as long as the effect of the invention is not impaired. Specifically, examples include methyl laurate, ethyl laurate, propyl laurate; methyl myristate, ethyl myristate, propyl myristate, methyl palmitate, ethyl palmitate, propyl palmitate, methyl stearate, ethyl stearate, propyl stearate, methyl hardened beef fat fatty acid, ethyl hardened beef fat fatty acid, propyl hardened beef fat fatty acid, methyl hardened fish oil fatty acid, ethyl hardened fish oil fatty acid, propyl hardened fish oil fatty acid, methyl coconut oil fatty acid, ethyl coconut oil fatty acid, propyl coconut oil fatty acid, methyl palm oil fatty acid, ethyl palm oil fatty acid, propyl palm oil fatty acid, methyl palm kernel oil fatty acid, ethyl palm kernel oil fatty acid, propyl palm kernel oil fatty acid, and the like. They may be used independently or in combination of two or more kinds.

As the fatty acid alkyl ester, a low iodine value is preferable in terms of color or odor. The iodine value of the fatty acid alkyl ester is preferably 0.5 or lower, more preferably 0.2 or lower.

Sulfonation of the fatty acid alkyl ester can be performed by contacting the fatty acid alkyl ester with a sulfonating gas, followed by reaction (sulfonation reaction).

Examples of the sulfonation reaction include known methods such as a thin film-type sulfonation method, batch type sulfonation method, a tank type reaction method, a film type reaction method, and a tube type gas-liquid mixed phase flow reaction method. The sulfonation reaction is not particularly limited as long as the effect of the invention is not impaired.

In the case of a tank type reaction method, for example, sulfonation can be performed by the following procedure. First, a fatty acid alkyl ester is charged into a reaction tank, and heated to form a starting liquid phase. Then, a sulfonating gas is introduced preferably at the constant flow rate into the starting liquid phase, and multiple bubbles occur from a gas sperger, and are dispersed into the starting liquid phase by rotation of a stirrer.

Examples of the sulfonating gas include SO₃ gas, fumed sulfuric acid, preferably SO₃ gas. The sulfonating gas is diluted with dehumidified air or inert gases such as nitrogen to about 3 to 20% by volume, preferably 4 to 10% by volume.

The amount of the sulfonating gas to be added is usually used at the proportion of an equivalent amount or more, preferably 1.0 to 2.0 moles, more preferably 1.1 to 1.5 moles with respect to 1.0 mole of fatty acid alkyl ester. When the amount of a sulfonating gas to be added is small, there is a problem in that the sulfonation reaction is not sufficiently performed. When the amount exceeds 2.0 moles, there is a problem in that the sulfonation reaction is radically performed to generate local heat, which results in coloration.

A reaction temperature of the sulfonation reaction may be a temperature where a fatty acid alkyl ester has a flowing property, generally, in terms of the melting point of a fatty acid alkyl ester, a high temperature not lower than 10° C. than the melting point described above is used, preferably 30 to 90° C., more preferably 40 to 80° C.

A reaction time is 5 to 120 seconds for a thin film type sulfonation method, and 10 to 180 minutes for a batch type sulfonation method.

A reaction mechanism where α-sulfo fatty acid alkyl ester is formed by sulfonation from fatty acid alkyl ester is described in Smith and Stirton: JAOCS vol. 44, P. 405 (1967) and Schmid, Baumann, Stein, Dolhaine: Proceeding of the World Surfactants Congress Munchen, vol. 2, P. 105, Gelnhausen, Kurle (1984) and H. Yoshimura: Oil Chemistry (JJOCS), 41, 10 (1992).

When the sulfonation reaction is performed, the reaction mechanism is performed as follows: one molecule of SO₃ is added to one molecule of a fatty acid alkyl ester to generate one molecule of adduct, one molecule of SO₃ is added to the one molecule of adduct to generate two molecules of adduct, and one molecule of SO₃ is removed from the two molecules of adduct to generate α-sulfo fatty acid alkyl ester. For example, when a fatty acid alkyl ester represented by formula (I) is specifically described as an example, one molecule of SO₃ is added to the fatty acid alkyl ester to generate one molecule of SO₃ adduct represented by formula (I′), one molecule of SO₃ is added to the one molecule of SO₃ adduct to generate two molecules of SO₃ adduct represented by formula (I″), one molecule of SO₃ is removed from the two molecules of SO₃ adduct to generate α-sulfo fatty acid alkyl ester represented by formula (II). In the specification, a series of reactions is called a sulfonation reaction.

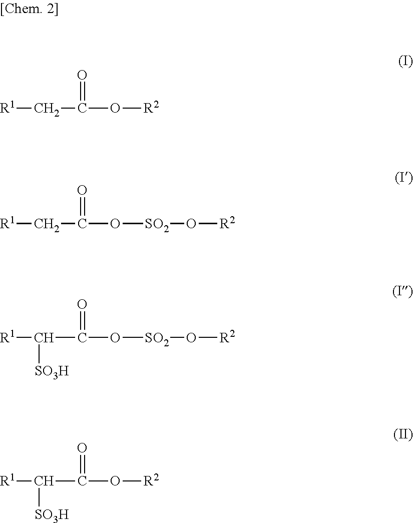

Wherein, R¹ and R² each have the same meaning as described above.

In Process (1), an aging treatment is preferably performed after sulfonation reaction.

The aging treatment is to maintain a predetermined temperature of a reactive product after sulfonation reaction.

The maintenance temperature (aging temperature) in an aging operation is preferably within a range of 70 to 100° C. When the aging temperature is 70° C. or higher, reaction is rapidly performed. When the aging temperature is 100° C. or lower, coloration can be inhibited at the time of aging.

The maintenance temperature (aging temperature) in the aging operation is dependent on the aging temperature, or the like, is generally about 1 to 120 minutes.

In the reaction mechanism described above, the reaction removing one molecule of SO₃ from two molecules of SO₃ adduct (I″) is a rate-limiting reaction, and therefore α-sulfo fatty acid alkyl ester (II) and two molecules of adduct (I″) are contained in the reactive product after sulfonation reaction. The aging treatment is necessarily added in order to obtain α-sulfo fatty acid alkyl ester, however, by the aging treatment, removing SO₃ from two molecules of adduct (I″) is promoted to improve the yield of α-sulfo fatty acid alkyl ester (II). Moreover, the side product α-sulfo fatty acid dialkali salt, or the like) derived from two molecules of adduct (I″) is inhibited to improve the yield of α-sulfo fatty acid alkyl ester salt.

Process (2)

In process (2), alcohol and hydrogen peroxide are added and reacted with the sulfonated product obtained by process (1).

Esterification reaction caused by alcohol and bleaching reaction caused by hydrogen peroxide are performed at the same time.

When two molecules of SO₃-adduct (I″) as an intermediate remain in the sulfonated product obtained by process (1), esterification of the alkoxy moiety of the two molecules of SO₃-adduct (I″) is performed by esterification reaction. That is to say, as described below, by reaction with alcohol (R³—OH), removal and ester exchange of SO₃ interposed in the alkoxy moiety of two molecules of SO₃ adduct (I″) are performed, and thereby α-sulfo fatty acid alkyl ester represented by formula (II′) is generated.

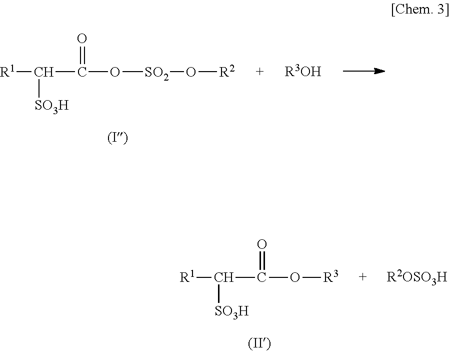

Wherein R¹ and R² each have the same meaning as described above, R³ is an alkyl group having 1 to 4 carbon atoms.

An alcohol used in the process, may be a straight or branched chain, preferably a monovalent alcohol. In particular, an alcohol having the same carbon atoms as those (for example, carbon atoms of R² in Formula (I)) of alcohol residue of starting fatty acid alkyl ester is preferable, an alcohol having the same alkyl group as that of the alcohol residue is more preferable. For example, when the starting material is a fatty acid methyl ester, methanol is preferably used.

An amount of the alcohol to be added is preferably 3 to 30% by mass, more preferably 15 to 25% by mass, with respect to the sulfonated product (100% by mass). When the amount is 3% by mass or more, esterification is sufficiently obtained. When the amount is 30% by mass or less, excessive alcohol should not be used, which is efficient.

Hydrogen peroxide is added as an aqueous solution. The concentration of hydrogen peroxide in the aqueous solution may be suitably adjusted with consideration for water volume in a reaction system, or reaction time, a reaction temperature, and it is usually about 30 to 40% by mass.

The amount of hydrogen peroxide to be added is preferably 0.1 to 10% by mass, more preferably 0.1 to 5% by mass, even more preferably 0.1 to 3.0% by mass, as the net component with respect to the sulfonated product (100% by mass).

In process (2), the reaction can be performed by adding alcohol and hydrogen peroxide with the sulfonated product, and keeping at the predetermined temperature for the predetermined time while it being suitably stirred.

A reaction temperature is preferably 70 to 100° C., more preferably 80 to 90° C.

A reaction time is preferably 30 to 120 minutes, more preferably 60 to 90 minutes.

Process (3)

In process (3), aqueous alkanolamine solution is added to the reactive product obtained by process (2) (hereinafter, referred to as “bleaching acid”) to a neutralized product having a pH of 2 to 6.5.

The pH of the bleaching acid is equal to or lower than 1, and thus a pH of the obtained neutralized product is adjusted within the range described above by adjusting an amount of the aqueous alkanolamine solution. Thereby, color deterioration can be inhibited at the time of condensation in process (4). The reason why the effect is obtained is not clear, but it is thought that an acidic pH results in stabilization of hydrogen peroxide and inhibition of color reversion.

The pH of the neutralized product is the pH at a neutralized temperature (temperature at the addition of the aqueous alkanolamine solution).

The pH of the neutralized product is preferably 6.1 or lower, more preferably 6 or lower, more preferably 5 or lower, in terms of having an excellent effect of color inhibition. Moreover, the pH is preferably 3 or higher, more preferably 4 or higher, with consideration for corrosivity to production facilities for the resultant α-sulfo fatty acid alkyl ester alkanolamine salt (hereinafter, referred to as α-sulfo fatty acid alkyl ester salt). When the pH is excessively reduced, the ester bond of the α-sulfo fatty acid alkyl ester salt is easily broken by hydrolysis.

For example, the neutralization can be performed by injecting the bleaching acid into a reaction tank, and adding and mixing an aqueous alkanolamine solution while it is kept at the predetermined temperature.

Moreover, neutralization may be performed by a loop neutralization method. In the method, one portion of the neutralized product (recycled product) is recycled in loop-type pipe (recycle loop), and therefore neutralization is performed by adding the recycled neutralized product to non-neutralized bleaching acid. According to such a method, not rapid neutralization but very mild neutralization can be performed, and hydrolysis of the generated α-sulfo fatty acid alkyl ester salt and side product in accordance therewith can be prevented.

A neutralization temperature is preferably 40 to 80° C., more preferably 40 to 60° C., and even more preferably 50 to 60° C. When the neutralization temperature is excessively reduced, there is a problem in that viscosity of the neutralized product increases, and thus manufacturing operations such as transfer and stirring are deteriorated. When the neutralization temperature is excessively increased, the resultant alkanolamine salt is easily hydrolyzed, and color deterioration or an increase of side products easily occurs.

Examples of the alkanolamine include one where 1 to 3 hydrogen atoms of ammonia (NH₃) is replaced with a hydroxylalkyl group having 1 to 3 carbon atoms.

Examples of the alkanolamine include ethanol amines such as monoethanolamine, diethanolamine, and triethanolamine. In particular, from the viewpoint of high effectiveness of the invention, triethanolamine is preferable.

An amount of the aqueous alkanolamine solution to be added in process (3) may be suitably adjusted with consideration for target pH of the neutralized product, molar concentration of the alkanol amine, or the like.

The molar concentration in the aqueous alkanolamine solution may be suitably adjusted with consideration for AI concentration of the neutralized product, it is not particularly limited, but usually 10 mol/L or less, preferably 1 to 7 mol/L.

“AI” means a compound having a function as a surfactant.

In the process, pH is within the range described above, and α-sulfo fatty acid alkyl ester in the bleaching acid obtained by process (2) is partially neutralized. Therefore, α-sulfo fatty acid alkyl ester salt and non-neutralized α-sulfo fatty acid alkyl ester are contained in the neutralized product. For example, when processes (1) and (2) are performed using a fatty acid alkyl ester represented by formula (I) as a starting material, α-sulfo fatty acid alkyl ester salt (IV) represented by formula (IV) as well as α-sulfo fatty acid alkyl ester represented by formula (II) are contained in the neutralized product in process (3).

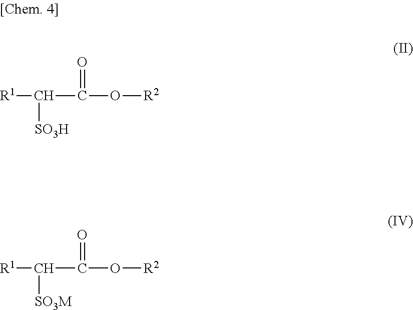

Wherein, R¹ and R² each have the same meaning as described above, M represents a quaternized alkanol amine.

Moreover, α-sulfo fatty acid dialkali salt (di-salt) as a side product in addition to α-sulfo fatty acid alkyl ester salt and α-sulfo fatty acid alkyl ester are contained in the neutralized product. Examples of the di-salt include a compound represented by formula (III).

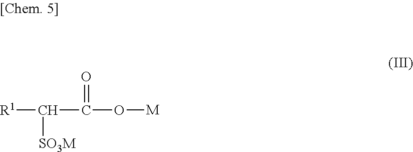

wherein, R¹ and M each have the same meaning as described above.

α-sulfo fatty acid alkyl ester or di-salt has a function as a surfactant in a similar manner to α-sulfo fatty acid alkyl ester salt.

Therefore, the AI concentration in the invention is determined as the total concentration of α-sulfo fatty acid alkyl ester salt, α-sulfo fatty acid alkyl ester, and α-sulfo fatty acid dialkali salt.

The AI concentration of the neutralized product is preferably 10 to 50% by mass. When the amount is 10% by mass or more, production efficiency increases. When the amount is 50% by mass or less, it is excellent in the effect of the invention. From the viewpoint of reduction of the neutralization addition, the AI concentration is preferably 30 to 50% by mass. The AI concentration of the neutralized product can be adjusted according to the additive amount and supply amount of water.

Process (4)

In process (4), the neutralized product obtained by process (3) is condensed to obtain an aqueous paste containing α-sulfo fatty acid alkyl ester alkanolamine salt.

Condensation and collection of alcohol can be performed by known methods, for example, by heating the neutralized product in the presence of normal pressure or reduced pressure.

A heating temperature is preferably 30 to 120° C., more preferably 50 to 110° C., and even more preferably 70 to 100° C. When the temperature is excessively increased, there is a problem in that decomposition of α-sulfo fatty acid alkyl ester salt or color deterioration occurs. When the temperature is excessively reduced, it takes time to condense, and therefore production efficiency is reduced.

The heating time is preferably 12 hours or shorter, more preferably 8 hours or shorter.

Heating can be performed using known heating devices, condensers, or the like. Examples of the heating devices include a hot plate, a thin film evaporator, a recycle flush, an evaporator, and an evaporating dish.

Condensation is preferably performed such that the water amount of the resultant condensed product (aqueous paste) is 10 to 40% by mass. The amount is more preferably 20 to 30% by mass. When the amount is within the range described above, there is an advantage in that the handling property is good.

Moreover, the AI concentration of the aqueous paste is preferably 60 to 90% by mass, more preferably 65 to 85% by mass in terms of flow properties. The AI concentration of the aqueous paste can be adjusted by adjustment of the AI concentration of the neutralized product before condensation, or water amount of the aqueous paste. On the other hand, the remaining alcohol is preferably 2% by mass counter AI or less.

Other Processes

A second neutralization process (5) in which a neutralization reaction is performed by adding the aqueous alkanolamine solution to the resultant aqueous paste may be performed after process (4).

When the pH of the neutralized product in process (3) is relatively low (for example, lower than a pH 6), there is a problem in that hydrolysis of α-sulfo fatty acid alkyl ester salt of the resultant aqueous paste occurs. Therefore, to improve the stability of the α-sulfo fatty acid alkyl ester salt, the pH after condensation is adjusted to a range in which hydrolysis does not easily occur. Moreover, even if the pH after condensation is higher than 6.5, color deterioration does not easily occur.

Here, the pH of the aqueous paste after the second neutralization process (5) is the pH at a temperature (neutralization temperature) at the time of the addition of the aqueous alkanolamine solution in a similar manner to the neutralized product described above.

The second neutralization process (5) can be performed in a similar manner to process (3).

It is preferable that the same alkanolamine as that in process (3) is used.

The pH of the aqueous paste after the second neutralization (5) is preferably 6.0 to 8.0, more preferably 6.5 to 7.5, in consideration of the effect and practicality.

When the second neutralization process (5) is performed, there are cases in which process (3) is called the first neutralization process.

Moreover, before process (4), an alcohol addition process (3′), which adds aliphatic alcohol as an additive, is provided, and thereby color deterioration after condensation cannot easily occur. The aliphatic alcohol means an alcohol having a hydrocarbon group having no aliphatic properties. The aliphatic alcohol may be saturated or unsaturated. Of these, a higher alcohol having 10 to 18 carbon atoms is preferable, and a higher alcohol having 12 to 14 carbon atoms is more preferable. In the specification, a higher alcohol represents an alcohol having 12 or more carbon atoms. Specifically, examples include lauryl alcohol, 1-tridecanol, myristyl alcohol, 1-pentadecanol, cetyl alcohol, 1-heptadecanol, stearyl alcohol, oleyl alcohol, linolyl alcohol, or the like.

When the aliphatic alcohol is 0.1 to 10% by mass with respect to AI, color deterioration at the time of condensation does not easily occur, which is preferable, and 1 to 5% by mass is more preferable.

After the aliphatic alcohol is added, the reactive solution may be heated. A heating temperature and heating time in which the neutralized product is homogeneously formed may be used. The temperature is usually 90° C. or lower, preferably, 80° C. or lower, and the time is 60 minutes or lower, preferably 30 minutes or lower.

It is more preferable that the alcohol addition process (3′) which adds the aliphatic alcohol is performed between processes (3) and (4).

The aqueous paste of the resultant α-sulfo fatty acid alkyl ester alkanolamine salt described above inhibits color deterioration when the neutralized product is condensed and has good color.

The aqueous paste may be used as a product as it is, and may be used in preparation of a composition containing surfactants such as liquid detergent components. Moreover, processes such as forming and assembling may be used.

### EXAMPLES

The invention will be more specifically described as Examples. Unless otherwise specified, “%” represents % by mass.

### Test Example 1

### 1. Sulfonation and Aging

To a 5 L four-neck flask, 2,150 g of fatty acid methyl ester having chain length C12 (12 carbon atoms) (PASTELL M-12, lot. No. P17059 manufactured by Lion Corporation) was charged, and an alcohol thermometer (measurement range 0 to 100° C.), a stirring blade (90 degrees paddle 20 mm×120 mm manufactured of Teflon (registered trademark)), and a blowing nozzle (inner diameter at the tip of nozzle: 2 mm) were set. This was immersed into a water bath, and the stirring blade was connected to a stirring motor. As a SO₃ evaporator, to a 1 L four-neck flask were set a Teflon (registered trademark) tube (inner diameter 2 mm, outer diameter 3 mm) from a pump (PUS16 manufactured by GL Science) and a nitrogen-introducing tube, and then the 1 L four-neck flask was set on a mantle heater. One neck of the flask was closed by a stopper and another neck was connected to the blowing nozzle of the 5 L four-neck flask by a glass tube, thereby forming a SO₃ evaporator.

The stirring blade was rotated at 250 rpm and the temperature of the water bath was adjusted to heat the methyl ester to 60° C. The voltage of the mantle heater was set to 80 V, and SO₃ gas (sulfur manufactured by Nisso metal) started to be fed to the SO₃ evaporator by a pump (flow rate: about 10 to 11 g/minutes), followed by starting to flow N₂ at 15 NL/minute (at this time, one neck of the 5 L four neck flask was connected to one end of a waste gas-discharging tube and the other end of the tube was set to a draft and used for discharging waste gas). 964.5 g of SO₃ gas was introduced into methyl ester for about 1.5 hours (SO₃/methyl ester 1.2 (molar ratio)). A temperature and water amount in the water bath was controlled and the liquid in the 5 L four flask was sulfonated at 60° C. to 85° C. Then, N₂ gas was stopped and a connected tube of the 5 L four neck flask was removed from the SO₃ evaporator.

The waste gas discharging tube was removed and replaced by a hole stopper, a blowing nozzle was removed and a Dimroth type reflux condenser was set, into which cooling water was flowed, and the reactive solution was heated at 80 to 85° C. for 30 minutes, after which the sulfonic acid was subjected to aging.

### 2. Preparation of Bleaching Acid

After aging, the temperature of the reactive solution was reduced to 60° C., a hole stopper was removed and a funnel was set, and 622.9 g (20% by mass with respect to reaction liquid amount) of methanol (manufactured by KANTO CHEMICAL CO., INC, Cica first grade) and 178.0 g (2% by mass of pure component hydrogen peroxide with respect to reaction liquid amount) of 35% aqueous hydrogen peroxide solution (manufactured by JUNSEI CHEMICAL CO., LTD., Junsei first grade) were added thereto. The funnel was removed and a hole stopper was set, the reactive solution was adjusted to 80 to 85° C., followed by reaction for 1 hour, esterification and bleaching of two molecules of SO₃ adduct were performed. Here, “reaction liquid amount” refers to an amount of injected methyl ester added to an amount of the introduced SO₃.

The resultant reactive product (bleaching acid of α-sulfo fatty acid (C12) methyl ester (hereinafter, referred to as C12MES) was cooled to about 60° C., a stirrer was stopped and the bleaching acid was taken away.

The bleaching acid was diluted with ethanol (manufactured by KANTO CHEMICAL CO., INC, Cica first grade) so as to be an ethanol solution having 5% by mass of solid content and when the color was measured by the following procedures, the measuring value was 15.

The ethanol solution was charged into a glass cell having an optical length of 40 mm, color (5% klett) was measured using a Klett-Summerson Photoelectric Colorimeter, model 900-3-equipped with a 420 nm blue filter.

### 3. Neutralization and Condensation

3-1. Preparation of Aqueous Paste Containing Sodium Salt of C12 MES (C12MES-Na)

About 1 kg of the bleaching acid was placed in a 10 L stainless steel vessel, in which a jet agitator manufactured by Shimazaki Mixing Equipment Co., Ltd was set, and vigorous stirring was carried out. 3 L of 0.5 mol/L NaOH aqueous solution at 50 to 60° C. was injected to be subjected to neutralization reaction. A pH meter was put in the reaction liquid so that the pH value was lower than 2. The pH meter used was a main body type pH71 and glass electrode type PH72SN-11, manufactured by Yokogawa Electric Corporation. Calibration of the pH meter was performed using a neutral phosphate pH standard solution (pH 6.86) and phthalate pH standard solution (pH 4.01).

While the pH electrode was immersed in liquid, aqueous 0.5 mol/L NaOH solution was added dropwise thereto, the pH was gradually increased, and sampling of about 300 g of solution was performed at pH2.0, pH4.0, pH6.0, and pH7.0.

After sampling, the samples were respectively moved to Petri dishes and mounted on a hotplate manufactured by Asahi Rika Co., Ltd., followed by condensation at a set temperature of the hotplate of 100° C. over 12 hours. When the water concentration was 20 to 25% by mass (the quantity was determined by Karl Fischer aquameter AQV-7 manufactured by HIRANUMA), condensation was stopped, to obtain an aqueous paste containing sodium salt of C12MES (C12MES-Na).

3-2. Preparation of Aqueous Paste Containing Monoethanolamine Salt of C12 MES (C12MES-MEA)

Neutralization and condensation were performed in a similar manner to (3-1. Preparation of aqueous paste containing sodium salt of C12 MES (C12MES-Na)) except that 0.5 mol/L of aqueous NaOH solution was changed to 0.5 mol/L of aqueous monoethanolamine solution (manufactured by KANTO CHEMICAL CO., INC, Cica first grade), to obtain an aqueous paste containing monoethanolamine salt of C12 MES (C12MES-MEA).

3-3. Preparation of Aqueous Paste Containing Triethanolamine Salt (C12MES-TEA) of C12 MES

Neutralization and condensation were performed in a similar manner to (3-1. Preparation of aqueous paste containing sodium salt of C12 MES (C12MES-Na)) except that 0.5 mol/L of aqueous NaOH solution was changed to 0.5 mol/L of aqueous triethanolamine solution (2,2′,2″-nitrilotriethanol manufactured by KANTO CHEMICAL CO., INC, Cica first grade), to obtain an aqueous paste containing triethanolamine salt of C12 MES (C12MES-TEA).

### 4. Measurement of AI Concentration and Color

AI concentration was measured by the following method with respect to each of the samples after condensation obtained by the (3. Neutralization and condensation).

200 mL of distilled water was added to each of the collected samples, which was transferred to a volumetric flask, followed by dilution with ion exchange water, which was added up to the gauge line to prepare a diluted solution. The diluted solution was obtained to a 5 mL Efton tube by a hole pipette, and 25 mL of methylene blue indicator and 15 mL of chloroform were added thereto, 5 mL of 4 mmol/L of benzethonium chloride solution was added thereto, followed by titration in 2 mmol/L aqueous sodium alkylbenzene sulfonate solution. A white plate was used as a background and an endpoint point was determined as a point when both layers had the same color. Similarly, a blank test was performed and from the difference of the titer and dilution degree, the AI concentration (% by mass) of each of the samples was calculated after condensation by the following formula.

Equation 1

AI concentration [% by mass]=(0.002×(B−A)×f×0.001×M)/(sample amount [g]×5/200)×100

A: volume [mL] of 2 mmol/L sodium alkylbenzene sulfonate solution required for the present test

B: volume [mL] of 2 mmol/L sodium alkylbenzene sulfonate solution required for blank test

f: titer of 2 mmol/L aqueous sodium alkylbenzene sulfonate solution

M: molecular weight of anionic surfactant

Each of the samples was diluted with ion exchange water so as to be an aqueous solution of AI concentration of 5% by mass, based on the calculated AI concentration, and the color (color after condensation) was measured by the following procedure.

Each aqueous solution was charged into a glass cell having an optical length of 40 mm, and color (5% klett) was measured using Klett-Summerson Photoelectric Colorimeter, model 900-3-equipped with a 420 nm blue filter.

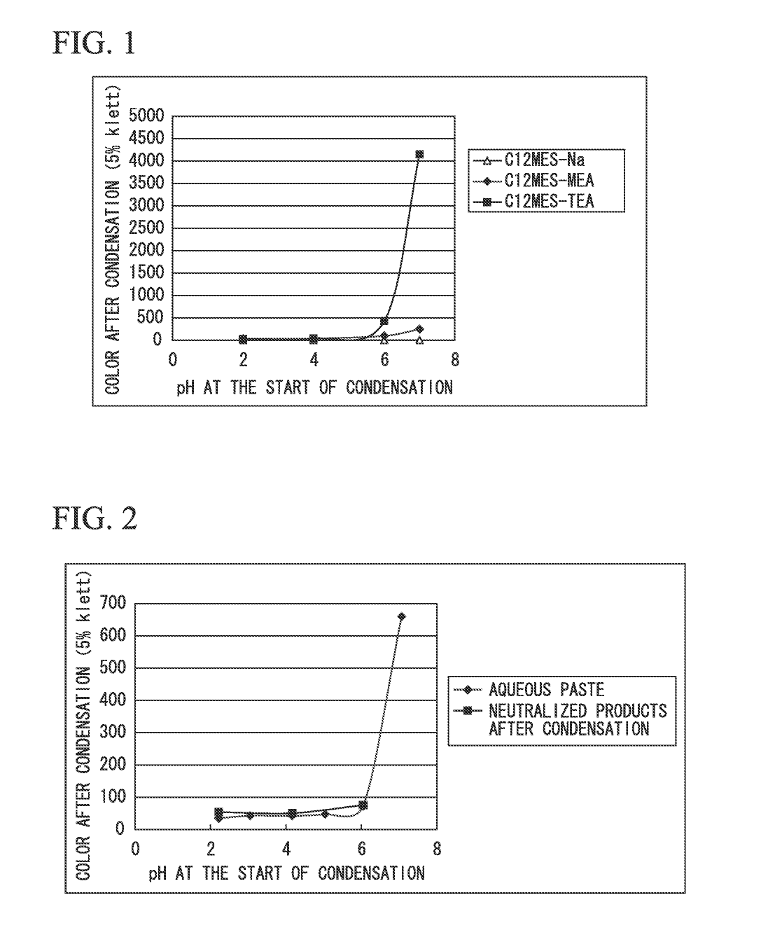

With respect to aqueous paste containing C12MES-Na, C12MES-MEA, C12MES-TEA, a graph was drawn using each measuring value (color after condensation (5% klett)) as a vertical axis, and the pH (pH of neutralized product) at the start of condensation as a horizontal axis. The graph is shown in FIG. 1. A measurement value of the color of each sample at each pH is shown in Table 1.

As shown in Table 1 and FIG. 1, in the case of sodium salt, the neutralized product of pH 7 was condensed and the color was not deteriorated, but in the case of alkanolamine salt, in particular triethanolamine salt, when the neutralized product of pH 7 was condensed, the color was deteriorated remarkably.

TABLE 1

pH

| | 2 | 4 | 6 | 7 |
|---|---|---|---|---|
| C12MES-Na | 5 | 5 | 5 | 6 |
| C12MES-MEA | 35 | 40 | 100 | 250 |
| C12MES-TEA | 31 | 42 | 430 | 4150 |

### Test Example 2

Sulfonation and aging were performed in a similar manner to [1. Sulfonation and aging] of Test Example 1 except that 3.000 g of fatty acid methyl ester of chain length C18 (18 carbon atoms) (PASTELL M-180, lot. No. P17059 manufactured by Lion Corporation) was charged thereto instead of fatty acid methyl ester of Chain C12 (12 carbon atoms), and 966.4 g of SO₃ was used.

Then, esterification and bleaching of two molecules of SO₃ adducts were performed in a similar manner to [2. Preparation of bleaching acid] of Comparative Example 1 except that 793.3 g of methanol and 226.7 g of 35% aqueous hydrogen peroxide solution were used. The color was measured in a similar manner to [2. Preparation of bleaching acid] of Test Example 1 and the color of the obtained bleaching acid was 35.

The bleaching acid was neutralized in a similar manner to [3-3. Preparation of aqueous paste containing triethanol amine salt of C12 MES (C12MES-TEA)] of Test Example 1, in which 0.5 mol/L of aqueous triethanolamine solution (2,2′,2″-nitrilotriethanol manufactured by KANTO CHEMICAL CO., INC, Cica first grade) was used. However, sampling was performed at pH 2.00, pH 3.00, pH 4.13, pH 5.00, pH 6.01, and pH 7.00. After one day, the pH of each of these samples was measured at 25° C., and pH values were pH 2.22, pH 3.05, pH 4.17, pH 5.04, pH 6.05, and pH 7.07.

Each of the samples was transferred to a Petri dish, and mounted on a hotplate (Asahi Rika Co., Ltd.,) followed by condensation at a setting temperature of the hotplate of 100° C. over 6 hours. When the water concentration was 20 to 30% by mass (the quantity was determined by Karl Fischer aquameter AQV-7 manufactured by HIRANUMA), the condensation was stopped, thereby obtaining an aqueous paste containing α-sulfo fatty acid alkyl ester alkanolamine salt of Chain length C18 (C18MES-TEA).

The AI concentration and color were measured in a similar manner to [4. Measurement of AI Concentration and color] of Test Example 1 with respect to each of the samples (aqueous paste (condensed product)) after condensation.

Then, each of the aqueous pastes was adjusted to 20% by mass of AI concentration with ion exchange water, followed by pH control to 7.0 with 0.5 mol/L aqueous triethanolamine solution, and then adjusted to 5% by mass of AI concentration with ion exchange water. The color (5% klett) of these neutralized products after condensation was measured in a similar manner to [4. Measurement of AI concentration and color].

With respect to each of the aqueous'pastes and the neutralized products after condensation, a graph using color (5% klett) as a vertical axis and the pH (the pH of the condensed product) at the start of condensation as a horizontal axis was drawn. The graph is shown in FIG. 2. Measurement values of pH at the start of condensation, that is to say the pH of each of the samples at 25° C. one day after neutralization are shown in Table 2.

TABLE 2

| | pH | | | | | |
|---|---|---|---|---|---|---|
| | 2.22 | 3.05 | 4.17 | 5.04 | 6.05 | 7.07 |
| C18MES-TEA | 35 | 43 | 43 | 48 | 73 | 660 |

As shown in Table 2 and FIG. 2, the neutralized product having a pH of 6.01 or lower has inhibited color deterioration after condensation, in addition, even when it was neutralized to a pH value of 7.0 after condensation, color deterioration was not shown.

### Test Example 3

### Examples 3-1 to 3-4, Reference Examples 3-5

Fatty acid methyl ester of chain length C14 (carbon atom 14) (trade name: PASTELL M-14, manufactured by Lion Corporation) was charged to a tank type reactor, SO₃ gas was blown into the reactive solution by nitrogen flow in an open system to sulfonate each of the methyl esters. The reaction temperature was 80° C. and the reaction molar ratio was SO₃/methyl ester of 1.2, in addition, the feed rate of SO₃ was about 10 g·min⁻¹. Then, aging was performed at 80° C. for 30 minutes to obtain α-sulfo fatty methyl ester. 20% by weight of methanol, 2% by weight of H₂O₂ (35% aqueous hydrogen peroxide solution 5.7% by weight) as net components were added to the resultant α-sulfo fatty methyl ester, followed by subjecting the resultant to reaction at 80° C. for 60 minutes and performing esterification and bleaching of two molecules of SO₃ adducts. 0.5 mol/L of aqueous monoethanolamine solution of 50° C. was added to the bleaching acid, and the pH of the sample was adjusted to 6.0 to obtain an aqueous α-sulfo fatty acid methylester monoethanol amine salt-containing solution. The resultant aqueous α-sulfo fatty acid methylester monoethanolamine salt-containing solution was charged into a sample bottle at a small amount and a higher alcohol (chain length C12 and C18, see Table 3) was added thereto, heated and dissolved (80° C., 15 minutes).

After cooling, the pH was adjusted to 6.0 by adding 0.5 mol/L of aqueous monoethanolamine solution, 30 g of each of the samples was transferred to a Petri dish, and mounted on a hot plate (set temperature 105° C.), followed by condensation for 8 hours, to obtain an aqueous paste containing α-sulfo fatty acid methyl ester monoethanolamine salt. With respect to each of the samples, AI after condensation and color were measured in a similar manner to [4. Measurement of AI Concentration and color] of Test Example 1. The result is shown in Table 3. As Comparative Example, AI after condensation and color were measured in a similar manner to [4. Measurement of AI concentration and color] of Test Example 1 with respect to a sample (Example 3-4) where higher alcohol was not added and condensed and a sample (Example 3-5) where lauryl acid was added and condensed instead of higher alcohol. In the Table, MES represents α-sulfo fatty acid methyl ester.

TABLE 3

| | | | | | Reference |
|---|---|---|---|---|---|
| | Example | Example | Example | Example | Example |
| | 3-1 | 3-2 | 3-3 | 3-4 | 3-5 |
| Chain length of MES | C14 | C14 | C14 | C14 | C14 |
| (carbon atoms) | | | | | |
| Additive | C₁₂H₂₅OH | C₁₂H₂₅OH | C₁₈H₃₇OH | None | Lauryl |
| | | | | | acid |
| Addition concentration of | 1 | 3 | 5 | — | 5 |
| additive | | | | | |
| (wt % with respect to AI) | | | | | |
| AI after condensation (wt %) | 83.4 | 82.6 | 79.9 | 89.2 | 79.8 |
| Color after condensation | 50 | 65 | 47 | 100 | 310 |
| (5% klett) | | | | | |

### Examples 3-6 to 3-7

An aqueous paste containing α-sulfo fatty acid methyl ester monoethanolamine salt was obtained in a similar manner to Example 3-4 except that fatty acid methyl ester of chain length C12 (trade name: PASTELL M-12, manufactured by Lion Corporation) and fatty acid methyl ester of the chain length C18 (trade name: PASTELL M-180, manufactured by Lion Corporation) were used. AI concentration and color after condensation are shown in Table 4

TABLE 4

| | Example 3-6 | Example 3-7 |
|---|---|---|
| MES Chain length (carbon atoms) | C12 | C18 |
| AI after condensation (wt %) | 80.2 | 79.6 |
| Color after condensation (5% klett) | 52 | 85 |

### INDUSTRIAL APPLICABILITY

According to the present invention, color deterioration can be inhibited at the time of condensation during preparation of alkanolamine salt of α-sulfo fatty acid alkylester.

1. A method for the preparation of α-sulfo fatty acid alkylester alkanolamine salt comprising:
sulfonating a fatty acid alkyl ester to obtain a sulfonated product,
reacting an alcohol and a hydrogen peroxide with the sulfonated product to obtain a reactive product,
adding an aqueous alkanolamine solution to the resultant reactive product to obtain a neutralized product having a pH of 2 to 6.5, and
condensing the neutralized product to obtain an aqueous paste containing α-sulfo fatty acid alkylester alkanolamine salt.

2. The method according to claim 1, further comprising a second neutralization process which adds an aqueous alkanolamine solution to the aqueous paste, followed by neutralization.

3. The method according to claim 1, further comprising adding aliphatic alcohol to the neutralized product.
